(19) United States
(12) Patent Application Publication
(10) Pub. No.: US 2023/0110841 A1
(43) Pub. Date: Apr. 13, 2023

(54) NONINVASIVE ARTERIAL PRESSURE WAVEFORM MEASUREMENT WITH CAPACITANCE AND OTHER SENSING

(71) Applicant: Cypress Semiconductor Corporation, San Jose, CA (US)

(72) Inventors: Richard SWEET, JR., San Diego, CA (US); Igor KOLYCH, Lviv (UA); Mykhaylo KREKHOVETSKYY, Lviv (UA); Igor KRAVETS, Lviv (UA); Oleksandr KARPIN, Lviv (UA); Andriy MAHARYTA, Lviv (UA)

(73) Assignee: Cypress Semiconductor Corporation, San Jose, CA (US)

(21) Appl. No.: 17/898,641

(22) Filed: Aug. 30, 2022

Related U.S. Application Data
(60) Provisional application No. 63/255,384, filed on Oct. 13, 2021.

(51) Int. Cl. A61B 5/021; A61B 5/277; A61B 5/00; A61B 5/256; A61B 5/257

(52) U.S. Cl. CPC A61B 5/02108; A61B 5/256; A61B 5/257; A61B 5/277; A61B 5/7285; A61B 2562/0214

(57) ABSTRACT

A system can include one or more electrodes; a sensor structure configured to position electrodes over a surface of a body that includes an artery. A capacitance sensing circuit can be coupled to the electrodes and configured to acquire capacitance values of the electrodes over a predetermined time period. The capacitance values can correspond to a distance between the body surface and the at least one electrode. Processor circuits can be configured to generate APW data from the capacitance values. Corresponding methods and devices are also disclosed.

### TECHNICAL FIELD

The present disclosure relates generally to biophysical sensors, and more particularly to noninvasive sensors for measuring arterial pressure waveforms, and related vital signs.

### BACKGROUND

When the heart ejects blood into the aorta it creates an arterial pressure wave that propagates down the arterial tree. It is the arterial pressure wave that is felt as the radial pulse. The resulting arterial pressure waveform (APW) can provide various vital signs and data, including heart rate, systolic blood pressure, diastolic blood pressure, to name but a few. Further, an APW waveshape can indicate numerous circulatory system conditions. An APW can be measured at any location on the body where an artery conveys blood.

Conventional approaches for acquiring APW and related data can include cuff-based tonometer/sphygmomanometer. However, cuff-based monitors are not convenient for taking continuous measurements, such as those needed for an APW. Another conventional approach involves invasive, internal arterial pressure sensors. While such internal sensors can provide continuous arterial pressure readings, they are highly invasive (require cannulation), and thus can be painful with physical effects (bruising).

It would be desirable to arrive at some way of continuously measuring arterial pressure that does not suffer from the drawbacks noted above.

### SUMMARY

Embodiments can include a biophysical sensor with one or more electrodes disposed over a body surface proximate an artery. Such electrodes can sense displacement in a skin surface caused by an arterial pressure wave to generate arterial pressure waveform (APW) data. In some embodiments, electrodes can be capacitive sensors. Changes in distance between the electrode and skin surface can result in capacitance changes, which can be used to generate an APW and/or related data. Such sensing can enable non-invasive and continuous sensing of an APW.

### BRIEF DESCRIPTION OF THE DRAWINGS

FIGS. 1A and 1B are diagrams showing a system and sensing operations according to embodiments.

FIGS. 2A to 2C are diagrams of sensor assemblies according to embodiments.

FIGS. 3A to 3C are diagrams showing capacitance sensing of an arterial pressure waveform (APW) according to an embodiment.

FIGS. 4A to 4C are diagrams showing sensor assemblies according to various embodiments. FIG. 4D is a diagram showing a sensor assembly utilizing mutual capacitance sensing according to an embodiment.

FIG. 5 is a side cross sectional view of a sensor assembly according to an embodiment.

FIGS. 6A and 6B are side cross sectional views of embodiments utilizing inductance sensing according to embodiments.

FIGS. 7A to 7C are diagrams showing sensor assemblies and corresponding test data according to embodiments.

FIG. 8 is a block diagram of an APW sensing system according to an embodiment.

FIG. 9 is a block diagram of an APW sensing system according to another embodiment.

FIGS. 10A and 10B are block diagrams of systems according to various embodiments.

FIG. 11 is a block diagram of a system according to another embodiment.

FIG. 12 is a block diagram of a system according to a further embodiment.

FIG. 13 are diagrams showing an APW sensing device as an integrated circuit package according to an embodiment.

FIGS. 14A and 14B are diagrams showing an APW sensing system according to another embodiment.

FIGS. 15A and 15B are diagrams showing an APW monitoring system and graphical user interface (GUI) according to an embodiment.

FIG. 16 is a flow diagram of a method according to an embodiment.

FIG. 17 is a flow diagram of a method according to another embodiment.

FIG. 18 is a flow diagram of a method according to another embodiment.

FIG. 19 is a diagram of a calibration method according to an embodiment.

### DETAILED DESCRIPTION

According to embodiments, a system can include a sensor structure which can position one or more electrodes over a body surface proximate an artery. As blood flows through the artery, displacement of the body surface can be detected by the electrode(s) to generate an arterial pressure waveform (APW) and/or related data.

In some embodiments, capacitance sensing can be used to detect such displacement, including self-capacitance of an electrode or mutual capacitance between electrodes. Other embodiments can use alternate sensing methods, including but not limited to resistance sensing or inductance sensing.

In some embodiments, a system can include multiple electrodes. A system can execute an initial scan of the electrodes to determine which electrodes have a high signal-to-noise ratio (SNR) with respect to the APW. A system can then acquire the APW and/or related data in one or more subsequent scans that use the high SNR electrodes and omit any low SNR electrodes.

Embodiments can include an array of electrodes.

Embodiments can include a reference electrode in addition to sensing electrodes. A reference electrode can be used in noise analysis and/or to sense conditions (e.g., temperature, noise) and adjust sense electrode value in response to the sensed conditions.

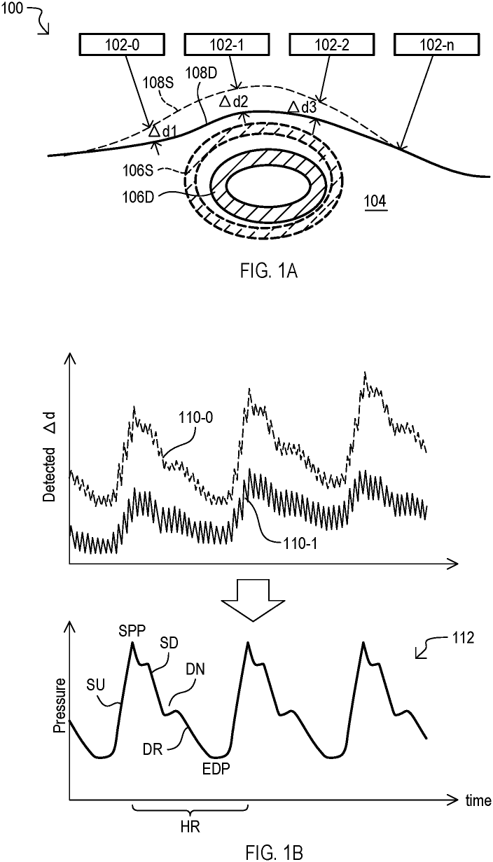

FIG. 1A is a diagram of a system 100 and corresponding operations according to an embodiment. A system 100 can include electrodes 102-0 to 102-n positioned over a surface of a body 104 near an artery. As blood is pumped an artery can change in size and/or position, shown as 106D and 106S. Changes in artery size/position 106D/S can cause changes in body surface position, shown as 108D/S. Changes in body surface 108D/S can introduce distance change (represented by Δd1, Δd2, Δd3) with respect to electrodes (102-0 to -n). This change in distance (e.g., Δd1, Δd2, Δd3) can be sensed by the electrodes (102-0 to -n). According to embodiments, such sensing by electrodes can take any suitable form, including changes in capacitance, inductance and/or resistance.

FIG. 1B shows show distance sensed by electrodes (two shown as 110-0 and 110-1), which can be capacitance values in some embodiments. Distance values 110-0/1 can be used to generate an APW 112 and/or APW related values. Such APW related values can include, but are not limited to: systolic upstroke (SU), systolic peak pressure (SPP), systolic decline (SD), dicrotic notch (DN), diastolic runoff (DR), end diastolic pressure (EDP), and heart rate (HR).

In this way, electrodes can non-invasively sense variations in a surface body generated by arterial blood flow to generate an APW and related data.

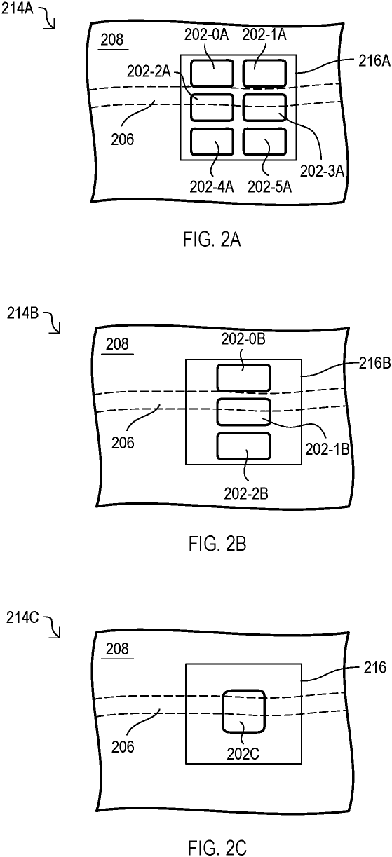

Embodiments can include one or more electrodes of any suitable configuration. FIGS. 2A to 2C show sensor assemblies according to some embodiments.

Referring to FIG. 2A, a sensor assembly 214A according to an embodiment is shown in a top plan view. A sensor assembly 214A can include a matrix of electrodes (202-0A to -5A) and a sensor structure 216A. A sensor structure 216A can position the array of electrodes (202-0A to -5A) over a body surface 208 near the location of an artery 206. A sensor structure 216A can take any suitable form, and can be rigid or flexible. It may or may not conform to a curve in a body surface 208. In some embodiments, electrodes (202-0A to -5A) can be arranged into an N × M matrix, where N and M are greater than one. While FIG. 2A presents a 2 × 3 matrix of electrodes (202-0A to -5A), other embodiments can include matrices of greater or smaller sizes. Further, electrode shapes can be different from one another and/or can be irregular in shape. Further, a matrix may not be regular, with one or more electrodes having a different spacing from other electrodes and/or electrodes not being arranged in regular columns and/or rows. A matrix of electrodes may advantageously provide multiple sources for detecting an APW.

Referring to FIG. 2B, a sensor assembly 214B according to another embodiment is shown in a top plan view. Sensor assembly 214B can include items like those of FIG. 2A, and can be subject to the same variations.as FIG. 2B. Sensor assembly 214B can include an array of electrodes 202-0B to 202-2B, which can be disposed in a direction different from that of the artery 206.

Referring to FIG. 2C, a sensor assembly 214C according to a further embodiment is shown in a top plan view. A sensor assembly 214C can include a single electrode 202C, which can have any suitable shape as described herein and equivalents.

In this way, embodiments can include one or more electrode of various shapes, sizes and configurations that can detect variations in a distance to a body surface caused by arterial blood flow.

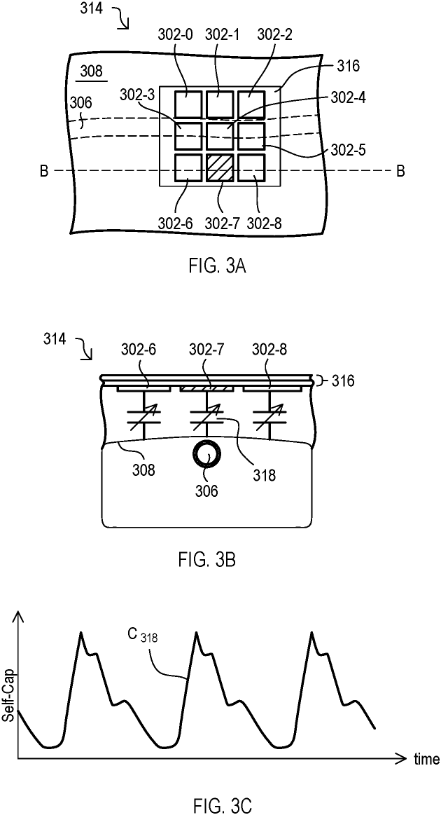

While embodiments can sense body surface movement using any suitable method, some embodiments can include capacitance sensing. FIGS. 3A to 3C show capacitance sensing according to one embodiment.

FIG. 3A is a top plan view of a capacitance sensor assembly 314 according to an embodiment. A sensor assembly 314 can include an array of capacitance sensing electrodes 302-0 to 302-8 and a sensor structure 316. In the embodiment shown, electrodes (302-0 to -8) can be arranged into a 3 × 3 matrix. Sensor structure 316 can position electrodes (302-0 to -8) over a body surface 308 near the location of an artery 306.

FIG. 3B is a side cross sectional view of sensor assembly 314 taken along line B-B of FIG. 3A. By operation of electrode structure 316, each of electrodes (three shown as 302-6 to 302-8) can be positioned over a body surface 308 by a distance. Such a distance can be sensed as a capacitance (e.g., 318).

FIG. 3C is a timing diagram showing a self-capacitance C₃₁₈ detected by an electrode. Such a self-capacitance C₃₁₈ can correspond to a displacement of body surface 308, which in turn, can correspond to an APW flowing through artery 306. A self-capacitance C₃₁₈ for all electrodes can be sensed, and from such data an APW and related data can be generated.

In this way, embodiments can use self-capacitance sensing by one or more electrodes disposed over a body surface to derive an APW and related data.

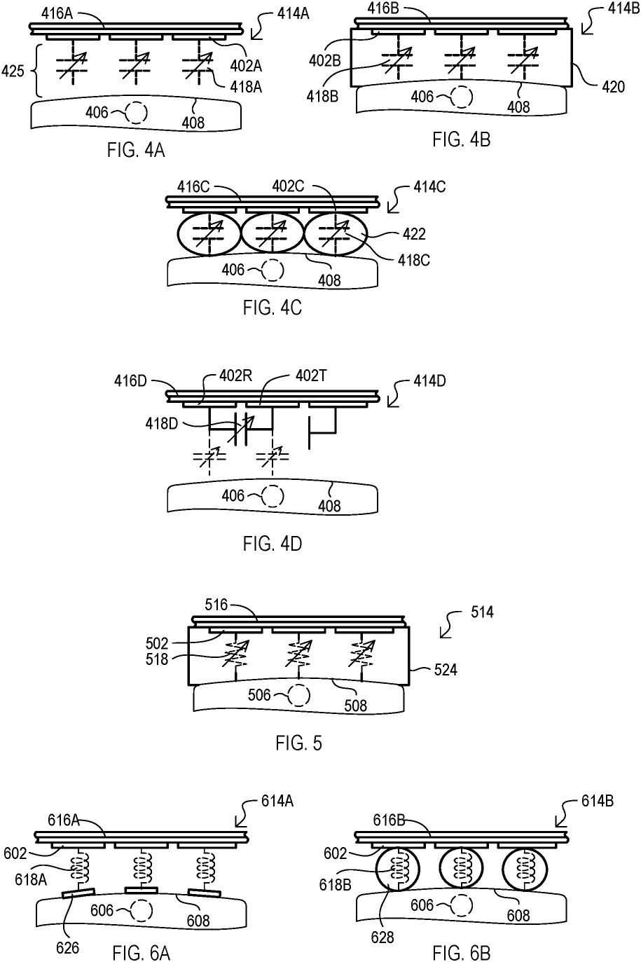

While FIGS. 3A to 3C show an APW sensor assembly according to one embodiment, alternate embodiments can take any suitable form. FIGS. 4A to 4C are side cross sectional views of sensor assemblies according to some alternate embodiments.

FIG. 4A is a side cross sectional view of a sensor assembly 414A with contactless self-capacitance sensing. Sensor assembly 414A can have electrodes (one shown as 402A) held over a body surface 408 with a sensor structure 416A. FIG. 4A shows how sensing can be contactless as an air gap 425 can exist between electrodes 402A and a body surface 408. FIG. 4A can be one version of that shown in FIGS. 3A to 3C.

FIG. 4B is a side cross sectional view of a sensor assembly 414B that includes capacitance sensing with contact measurement. A sensor assembly 414B can include a high permittivity (hi-k) compressible material 420 positioned between electrodes (one show as 402B) and a body surface 408. Sensor assembly 414B can be conceptualized as executing a pressure measurement. In response to artery blood flow, a position of a body surface 408 can change, pressing against compressible material 420. Such pressure changes can result in self-capacitance changes 418B over time at electrodes 402B. Unlike other approaches, such as resistance measurements, pressure sensing like that of FIG. 4B can be advantageously insensitive to changes in skin conductivity, such as that resulting from sweating or the like.

FIG. 4C is a side cross sectional view of a sensor assembly 414C with volume displacement measurement. A sensor assembly 414C can include high permittivity compressible volumes 422 positioned between electrodes (one show as 402C) and a body surface 408. Sensor assembly 414C can also be conceptualized as executing a pressure measurement. In response to artery blood flow, a body surface 408 can press against volumes 422. Such pressure changes can result in self-capacitance changes 418C over time at electrodes 402C.

In this way, embodiments can execute self-capacitance sensing in various ways to determine an APW and related data.

While embodiments can include sensors assemblies that utilize self-capacitance, other embodiments can sense a mutual capacitance between electrodes. An example of such an embodiment is shown in FIG. 4D.

FIG. 4D is a side cross sectional view of a sensor assembly 414D utilizing mutual capacitance sensing. A sensor assembly 414D can include electrodes (two shown as 402R and 402T) positioned over a body surface 408 by sensor structure 416D. A sensor assembly 414D can sense a mutual capacitance 418D between electrodes (402R/T). It is understood that in some embodiments such sensing can be dynamic, switching which pair of electrodes is sensed in a scanning sequence. A mutual capacitance 418D can vary in response to distance to a body surface 408, which can vary in response to blood flow through artery 406.

In this way, embodiments can execute mutual capacitance sensing to determine an APW and related data.

While embodiments can include sensor assemblies that utilize capacitance measurements of different types, other embodiments can utilize other forms of sensing with electrodes. FIGS. 5 to 6B show examples of such various alternate embodiments.

FIG. 5 is a side cross sectional view of a sensor assembly 514 having resistance sensing to determine an APW or related data. A sensor assembly 514 can include electrodes (one shown as 502) and a compressible conductive material 524 positioned between the electrodes 502 and a body surface 508. In some embodiments, a material 524 can be an anisotropic rubber material, that has a resistance that changes in a vertical direction as it is compressed (more than it changes in a horizontal direction). A sensor assembly 514 can sense a resistance (e.g., 518) between electrodes 502 and a body surface 508. Thus, a sensor assembly 514 can be another example of contact sensing, like that shown in FIG. 4B.

In this way, embodiments can execute resistance sensing to determine an APW and related data.

FIG. 6A is a side cross sectional view of a sensor assembly 614A having inductance sensing. A sensor assembly 614A can include electrodes (one shown as 602), which can take a form suitable for sensing an inductance. In the embodiment shown, body electrodes 626 can be placed on a body surface. Variations in inductance 618A can correspond to movement of body surface caused by blood flowing through artery 606. Such inductance variations 618A can be used to determine an APW and related data.

FIG. 6B is a side cross sectional view of a sensor assembly 614B having inductance sensing according to another embodiment. A sensor assembly 614B can include electrodes (one shown as 602), which can take a form suitable for sensing an inductance. In addition, conductive compressible volumes (one shown as 628) can be disposed between electrodes 602 and a body surface 608. As a body surface 608 move in response to blood flow in artery 606, volumes 608 can change in shape, and thus change in inductance 618B. Such inductance changes 618B can be used to generate an APW and related data.

In this way, embodiments can execute inductance sensing to determine an APW and related data.

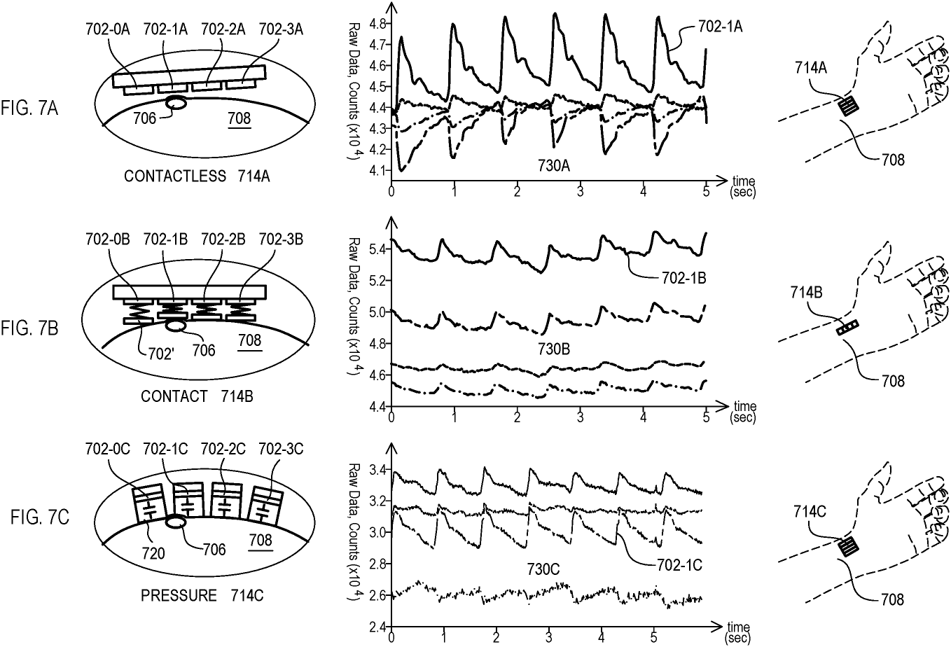

FIGS. 7A to 7C are diagrams showing test data for various embodiments. Each of FIGS. 7A to 7C shows a different type of sensor assembly 714A to 714C, a representation of test data 730A to 730C for the sensor assembly, and a representation of a sensor assembly 714A to 714C on a subject body 708.

FIG. 7A shows an example of a contactless type sensor assembly 714A. Electrodes 702-0A to 702-3A are positioned above a surface of a body 708 in proximity to an artery 706. Representative test data 730A shows how different electrodes can generate different data. In the embodiment shown, data can be count values generated by the analog-to-digital conversion of a sensed value, such as a capacitance (or resistance or inductance). In the embodiment shown, test data for an electrode 702-1A generates the most dynamic data waveform. This can arise from the movement of a body surface being greatest below this electrode. Some or all data sets from the various electrodes can be used to generate an APW and related data.

FIG. 7B shows an example of a contact type sensor assembly 714B. Electrodes 702-0B to 702-3B can include portions (one shown as 702′) positioned on a surface of a body 708 in proximity to an artery 706. In the embodiment shown, test data waveforms 730B can be count values generated by the analog-to-digital conversion of a sensed value, such as a capacitance (or resistance or inductance).

FIG. 7C shows an example of a pressure type sensor assembly 714C. Electrodes 702-0C to 702-3C can include portions (one shown as 720) that can sense pressure from a body surface. While pressure sensing portion 720 is shown to generate a variation in capacitance, alternate embodiments can generate changes in resistance or inductance. Waveforms 730C can sense pressure differences sensed by electrodes (702-0C to -3C).

While a sensor assemblies 714A to 714C can be located at any suitable location on a body 708, FIGS. 7A to 7C show sensor assemblies positioned on a wrist. As will be described in more detail below, in some embodiments, data from some sensors can be omitted from analysis according to its quality (e.g., signal-to-noise ratio, SNR).

In this way, variations in a body surface position can be sensed by multiple electrodes over time to arrive at an APW and related data.

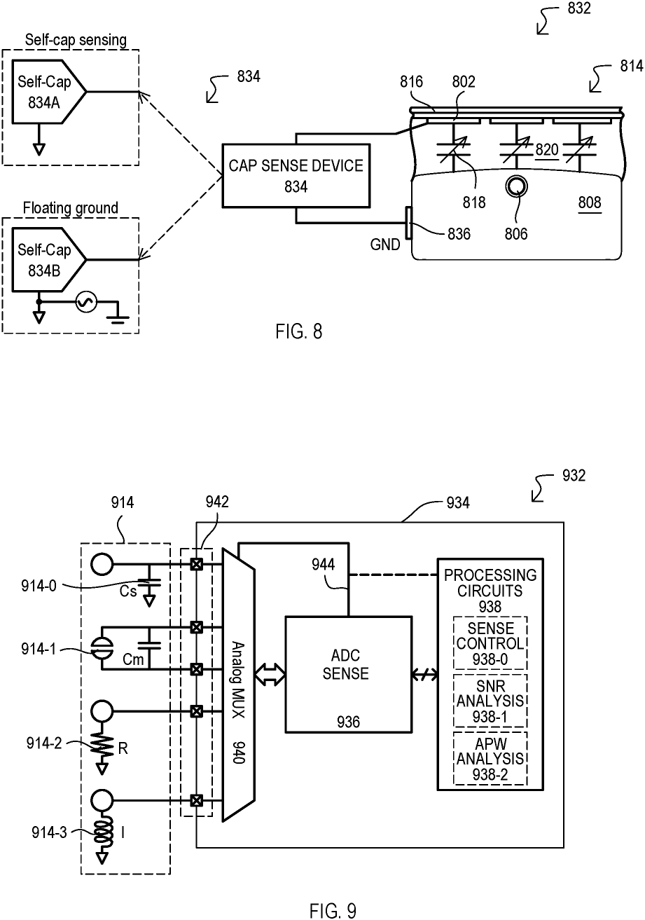

FIG. 8 is a block diagram of a capacitance APW sensing system 832 according to embodiments. A system 832 can include a sensor assembly 814 and a capacitance sensing device 834. A sensor assembly 814 can include a sensor structure 816 and capacitance sensing electrodes (one shown as 802). In the embodiment shown, a compressible material (e.g., hi-k material) 820 can be disposed between electrodes 802 and a surface of a body 808 that includes an artery 806. However, alternate embodiments can include any other suitable capacitance sensing structure. Electrodes 802 can detect variations in capacitance 818 generated by changes in a surface of body 808 caused by blood flow through artery 806.

A capacitance sensing device 834 can sense a self-capacitance of electrodes 802 over time, and from such data, derive an APW and/or related data. In some embodiments, a capacitance sensing device 834 can include a ground connection 836 to a body 808. A capacitance sensing device 834 can take any suitable form, and FIG. 8 shows two of many possible configurations. In one configuration, a capacitance sensing device 834A can excite each electrode 802 when determining a self-capacitance at such an electrode 802. In another configuration, a capacitance sensing device 834B can excite a device ground to some voltage when determining a self-capacitance at electrodes 802.

In this way, embodiments can utilize various self-capacitance sensing method to derive an APW and related data.

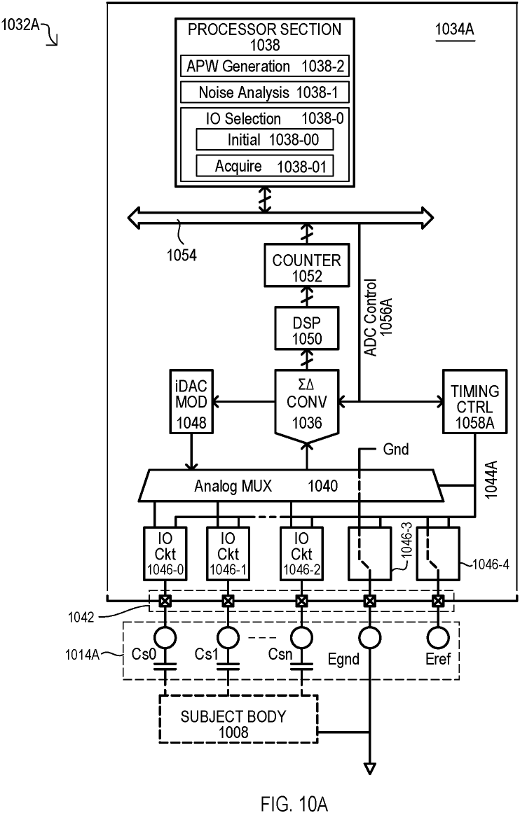

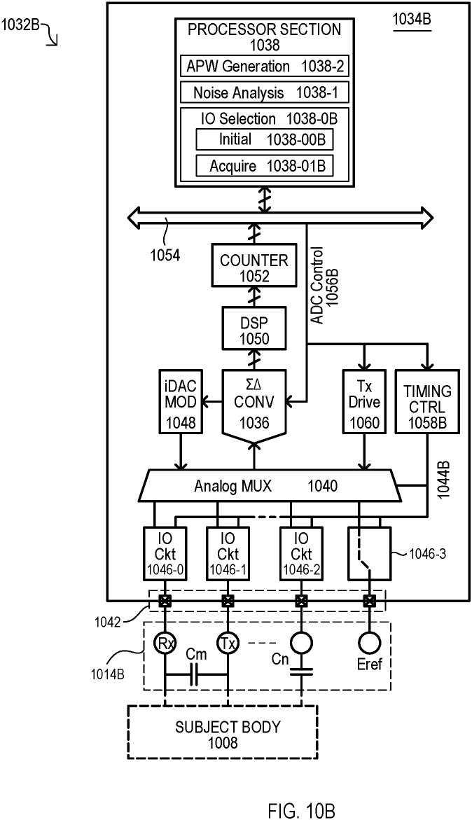

FIG. 9 is a block diagram of an APW sensing system 932 according to another embodiment. A system 932 can include a sensing device 934 and a sensor assembly 914. A sensor device 934 can include analog-to-digital converter (ADC) sense circuits 936, processor circuits 938, an analog multiplexer (MUX) 940 and input/outputs (IOs) 942. ADC sense circuits 936 can convert input values/signals (e.g., current, voltage) received from analog MUX 940 into digital values for processing by processing circuits 938. ADC sense circuits 936 can include any suitable ADC circuits, including but not limited to: “flash” ADCs, sigma-delta ADCs, or a successive approximation register (SAR) type ADC.

Analog MUX 940 can selectively connect IOs 942 to ADC sense circuits 936 in response to control signals 944 generated from ADC sense circuits 936 and/or processing circuits 938. IOs 942 can be connected to sensor electrodes, or the like, used for detecting artery body surface movement in response to artery as described herein.

Sensor assembly 914 can include one or more sensors (914-0 to 914-3) Sensors (914-0 to -3) can detect surface movement in response to artery blood flow. Sensors (914-0 to -3) can take the form of any of those described herein, including but not limited to self-capacitance sensors 914-0, mutual capacitance sensors 914-1, resistance sensors 914-2 and/or inductance sensors 914-3.

Processing circuits 938 can include any suitable circuits for executing various sense functions, including but not limited to: one or more processors (with corresponding memory), custom logic circuits, programmable logic circuits, or combinations thereof. Processing circuits 938 can provide sense control functions 938-0, signal analysis functions 938-1 and APW analysis functions 938-2. Sense control functions 938-0 can control operations of ADC sense circuits 936 and/or analog MUX 940. SNR analysis function 938-1 can determine a SNR for data received from each electrode. Such a feature can enable electrodes with lower SNRs to be excluded from analysis that generates an APW or related data. APW analysis function 938-2 can receive data generated by ADC sensing circuits 936, and determine an APW and/or related data therefrom. In some embodiments, APW analysis function 938-2 can use determinations from SNR analysis function (e.g., to exclude low SNR data).

In this way, an APW capacitance sensing system can include ADC converting circuits and digital processing circuits to generate APW and related data.

FIG. 10A is a block diagram of a system 1032A according to another embodiment. A system 1032A can use self-capacitance sensing and a sigma-delta analog-to-digital conversion to generate an APW and related data. A system 1032A can include an APW sensing device 1034A and sensor assembly 1014A. APW sensing device 1043A can include IOs 1042, IO circuits 1046-0 to 1046-4, analog MUX 1040, sigma-delta (ΣΔ) converter circuit 1036, a current digital-to-analog converter (iDAC) (current source) modulator 1048, timing control circuit 1058A, digital signal processing (DSP) circuits 1050, counter 1052, processor section 1038 and digital bus 1054. IOs 1042 can connect to a sensor assembly 1014A. IO circuits (1046-0 to -4) can enable various connections between IOs 1042 and analog MUX 1040. Such connections can be input connections (e.g., to read current/voltages) and/or output connections (e.g., driving currents/voltages). Timing of such connections can be established by timing control circuit 1058A. In the embodiment shown, IO circuit 1046-3 can enable a ground connection (Egnd) to sensor assembly 1014A and IO circuit 1046-4 can enable a connection to a reference electrode (Eref). As will be described herein, a reference electrode Eref can be used by system 1032A to determine sensing conditions (e.g., temperature, noise). IO circuits (1046-0 to -2) can be connected to other components, such as sampled capacitances (Cs0 to Csn).

Analog MUX 1040 can selectively connect IO circuits (1046-0 to -4) to various other circuits of APW sensing device 1034A. It is understood that analog MUX 1040 can provide both input paths from and output paths to IO circuits (1046-0 to -4). In some embodiments, paths through analog MUX 1040 can be bidirectional. Path switching of analog MUX 1040 can be controlled by timing control circuit 1058A.

ΣΔ converter circuit 1036 can execute ΣΔ type ADC operations to generate a bit stream that varies according to a detected capacitance (which can take the form of an analog current or voltage). iDAC modulator 1048 can modulate a current at a sampled node in response to control signals from ΣΔ converter circuit 1036 during a conversion operation. DSP circuits 1050 can process digital data provided by ΣΔ converter circuit 1036. DSP circuits 1050 can include any suitable operations according to conversion method, including but not limited to digital filtering and/or scaling functions. A counter 1052 can generate digital count values representative of a sampled self-capacitance over time. ΣΔ converter circuit 1036 and timing and control circuit 1058A can receive control signals 1056A which can be received from processor section 1038 via a digital bus 1054.

Processor section 1038 can include processing circuits as described herein and equivalents. Processor section 1038 can execute an IO selection function 1038-0, a noise analysis function 1038-1 and an APW generation function 1038-2. An electrode selection function 1038-0 can include initial operations 1038-00 and acquisition operations 1038-01. Initial operations 1038-00 can control access to IOs 1042 to sense values from all relevant electrodes. Such an operation 1038-00 can include sensing values at electrodes that sense a self-capacitance (e.g., Cs0 to Csn). Such an operation can also sense values at a reference electrode (e.g., Eref). Based on values generated by an initial sensing operation 1038-00, a noise analysis function 1038-1 can determine which IOs (i.e., sense electrodes) provide a highest quality signal (e.g., have the highest SNR, or an SNR above a predetermined threshold). In some embodiments such an action can utilize noise or condition data from a reference electrode (Eref).

Once a quality of self-capacitance data has been determined for each IO 1042, an acquisition operation 1038-01 can acquire data from the high quality (e.g., high SNR) IOs. Such an operation 1038-01 can acquire data values used to generate an APW. In some embodiments, such an operation can acquire data for no less than two waveforms of an APW. APW generation function 1038-2 can generate an APW and/or related data. Such a function 1038-2 can utilize data values corresponding to one or more fully sampled APW time periods.

In this way, a system can utilize ΣΔ conversion operating on high quality self-capacitance electrodes to arrive at an APW and related data.

FIG. 10B is a block diagram of a system 1032B according to another embodiment. A system 1032B can use mutual capacitance sensing and ΣΔ analog-to-digital conversion to generate an APW and related data. A system 1032B can include items like those of FIG. 10A, and such like items have the same reference characters, and can operate in the same general fashion.

System 1032B can differ from that of FIG. 10A in that IOs 1042 and sensor assembly 1014B can be configured for mutual capacitance sensing. While any mutual capacitance method can be employed, in the embodiment of FIG. 10B, in a conversion operation, one electrode can be selected as a transmit electrode (Tx), and can be driven with a signal by a transmit driver circuit 1060 via analog MUX 1040 and the corresponding IO circuit (1046-1). Further, another electrode can be selected as a receiving electrode (Rx). By operation of analog MUX 1040 a mutual capacitance Cm can be sensed between the Rx and Tx electrodes by ΣΔ converter circuit 1036. In some embodiments, a system 1032B can cycle through various pairs of electrodes to determine multiple mutual capacitance values for an electrode array/matrix. Accordingly, timing and control circuit 1058B can select pairs of electrodes, enabling one electrode to be driven as a transmit electrode, and one to act as a receiving electrode. In some embodiments, such an action can be controlled by ADC control signals 1056B provided from processor section 1038.

In the embodiment of FIG. 10B, initial operations 1038-00B can sense multiple mutual capacitances, as described herein. Acquisition operation 1038-01 can select electrode pairs having a high quality (e.g., high SNR) as determined from their sensed mutual capacitance.

In this way, a system can utilize ΣΔ conversion operating on high quality mutual capacitance sensing electrodes to arrive at an APW and related data.

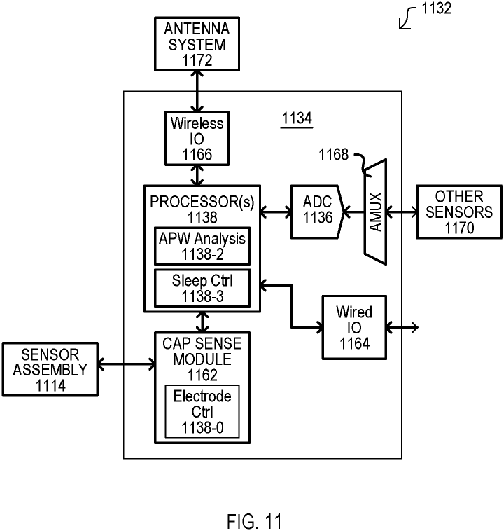

FIG. 11 is a block diagram of another system 1132 according to an embodiment. A system 1132 can communicate with other devices via a wired or wireless connection to relay an APW and/or related data. A system 1132 can include an APW sensing device 1134, a sensor assembly 1114, an antenna system 1172, and optionally, other analog sensors 1170. APW device 1134 can include processor circuits 1138, a capacitance sense module 1162, ADC circuit 1136, an analog MUX 1168, and wired IO circuits 1164. Processor circuits 1138 can perform various capacitance sensing APW functions as described herein or equivalents, including APW analysis 1138-2 (i.e., deriving APW data from capacitance sensing values). In the embodiment shown, processor circuits 1138 can also include sleep control circuits 1138-3. Sleep control circuits 1138-3 can control capacitance APW sensing operation to limit power consumption. As but one example, sleep control circuits 1138-3 can establish a periodicity at which APW measurements are taken. in In some embodiments, such a periodicity can be programmable by a user.

A capacitance sensing module 1162 can include circuits specifically designed for capacitance sensing, including self-capacitance sensing or mutual capacitance sensing. In some embodiments, capacitance sensing module 1162 can include electrode/IO selection circuits, ADC circuits and signal conditioning circuits (e.g., filters) as described herein or equivalents. Capacitance sensing module 1162 can be connected to a sensor assembly 1114 which can the form of any of those described herein or equivalents.

ADC circuit 1136 and analog MUX 1140 can enable additional sensing capabilities. In some embodiments, ADC circuit 1136 is not used in capacitance sensing by capacitance sensing module 1162. Analog MUX 1140 can enable connection to various other sensors 1170 of a system 1132. Such other sensors can take any suitable form including those described herein, as well as others (e.g., oxygen sensors, movement sensors, blood glucose sensors). Wired IO circuits 1164 and wireless IO circuits 1166 can enable communication with the APW sensing device 1134, including the output of APW data and/or the input of control values to control APW sensing. An antenna system 1172 can be included to enable wireless transmission reception.

In this way, a system can sense APW data and include additional sensor inputs as well as wired and/or wireless communication of APW data and/or APW sensing control data.

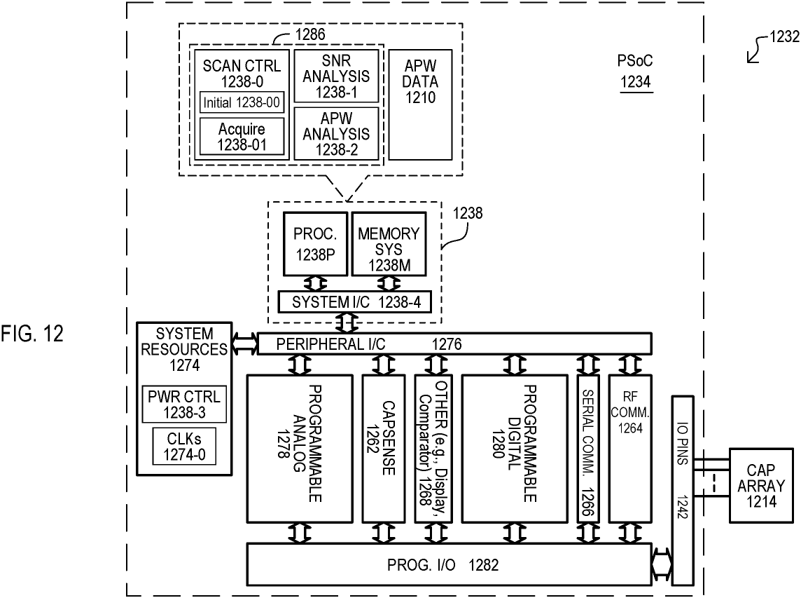

FIG. 12 shows a system 1232 according to a further embodiment. A system 1232 can include a sensor assembly 1214 and programmable system on chip (SoC) 1234 configured as an APW sensing device 1234. A sensor assembly 1214 can take the form of any of those described herein or equivalents.

Programmable SoC 1234 can include processing circuits 1238, system resources 1274, peripheral interconnect 1276, programmable analog circuits 1278, capacitance sense circuits 1262, other fixed circuits 1268, programmable digital circuits 1280, communication circuits 1266, RF communication circuits 1264, programmable IOs 1282, and IO pins 1242. Processing circuits 1238 can include a processor section 1238P and memory section 1238M connected to one another by a system interconnect 1238-4. Processor section 1238M can include one or more processors. A memory section 1238M can include one or more memory circuits, including volatile and/or nonvolatile memory circuits. In some embodiments, a memory section 1238M can store APW data 1210 as well as instructions executable by processor section 1238P to provide various functions 1286. Such functions 1286 can include, but are not limited to: scan control 1238-0, SNR analysis 1238-1 and APW analysis 1238-2. Scan control functions 1238-0 can control scanning of sensing electrodes as described herein, including an initial scan 1238-0 used to determine which electrodes provide high quality (e.g., high SNR) data, and an acquisition scan 1138-01, which can exclude electrodes with low quality data.

System resources 1274 can provide or control various system resources of the SoC 1234, and can include power control 1238-3 and timing clocks 1274-0. Power control 1238-3 can control power to the system 1232, including placing APW sensing device 1234 into a sleep mode between capacitance sensing operations. Peripheral I/C 1276 can enable connection between processing circuits 1238 and other sections of the device 1234. Programmable analog circuits 1278 can include analog circuit elements that can be configured with configuration data. Capacitance sense circuits 1262 can be connected to sensor assembly 1214 via programmable IO 1282, and can execute capacitance sense functions with the sensor assembly 1214. Other fixed circuits 1284 can include circuits having various fixed functions, including but not limited to display drivers and analog comparators.

Programmable digital circuits 1280 can include digital circuits configurable in response to configuration data. In some embodiments, programmable digital circuits 1280 can include, or be configured into, digital filters and/or counters that can be included in capacitance sensing operations. Communication circuits 1266 can enable communications with the system 1232, and can include any suitable interface, including one or more serial interfaces. Communication circuits 1266 can be connected to IOs 1242. RF communication circuits 1264 can enable wireless communications with the system 1232 according to one or more wireless protocols, including but not limited to Bluetooth (including BLE), IEEE 802.11 wireless protocols and/or cellular protocols. RF communication circuits 1264 can enable a device 1234 to wirelessly communicate with other devices, including receiving configuration data for establishing capacitance sensing parameters, as well as transmitting APW and related data. RF communication circuits 1264 can be connected to one or more antenna systems (not shown) via IOs 1242 or other dedicated IOs.

In this way, a system can include a controller device with configurable analog circuits and/or configurable digital circuits. Such an arrangement can enable a common architecture to accommodate sensors in addition to capacitance sensors. Signal paths and processing can be configured for capacitance and other sensors. Signal processing can be hardware accelerated with programmable digital circuits according to sensor type.

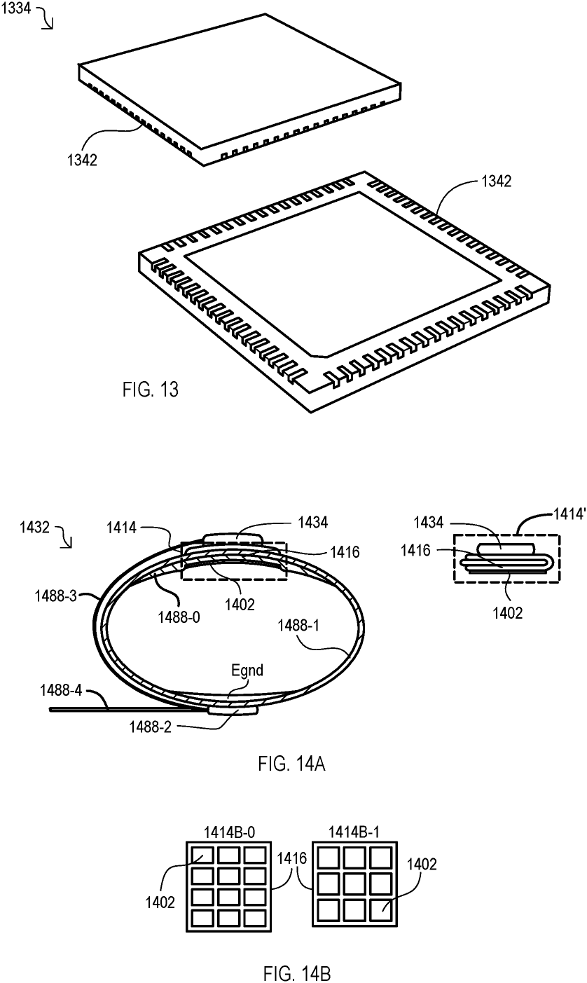

While embodiments can include systems with various interconnected components, embodiments can also include unitary APW sensing devices which can connect to one or more APW sensing assemblies one or more IOs. FIG. 13 shows a packaged single chip APW sensing device 1334. Device 1334 can include circuits like those shown in any of FIGS. 8-12, or equivalents. Device 1334 can include IOs (one shown as 1342), which can be configured for electrode sensing of an APW according to any of the techniques described herein, or equivalents. Such circuits can be formed in a single integrated circuit package and/or a same integrated circuit substrate.

However, it is understood that a device according to embodiments can include any other suitable integrated circuit packaging type, as well as direct bonding of a device chip onto a circuit board or substrate.

FIGS. 14A and 14B are diagrams showing an APW sensing system 1432 according to another embodiment. APW sensing system 1432 can be a bracelet wearable by person to sense an APW and/or related data of that person. A bracelet system 1432 can include an APW sensing device 1434, sensor assembly 1414, frame 1488-0, bracelet 1488-1, ground electrode Egnd, and connector 1488-2. A sensor assembly 1414 can include an electrode structure 1416 with electrodes 1402 in communication with APW sensing device 1434. APW sensing device 1434 and sensor assembly 1414 can take the form of any of those described herein and equivalents, including contactless and contact configurations. Sensing can include capacitance, resistance and inductance sensing.

Bracelet 1488-1 can enable electrodes 1402 to be placed over an artery of a person for APW sensing. Such an arrangement can enable continuous and/or periodic sensing of an APW that is non-invasive and convenient for a subject. In the embodiment shown, a frame 1488-0 can be included to hold a sensor assembly 1414. Ground electrode Egnd can be included on an inner surface of bracelet 1488-1 to provide a ground contact with the body being sensed. Ground electrode Egnd can have a connection to an IO of APW sensing device 1434 (not shown). An APW device sensing device 1434 can be in communication with a connector 1488-2, which can provide a connection to other devices. In the embodiment shown, a data connection 1488-3 can be a wired connection between APW sensing device 1434 and connector 1488-2, but other embodiments can include a wireless connection. Similarly, a connector 1488-2 can be in communication with other systems via an external connection 1488-4, which can be wired or wireless.

FIG. 14A also shows an alternate sensor assembly 1414′. Alternate sensor assembly 1414′ can be folded as compared to that shown as 1414, for a more compact structure.

FIG. 14B are diagrams showing sensor assemblies 1414B-0 and 1414B-1 that can be included in a system like that of FIG. 14A. Sensors assemblies 1414-B0/1 can include electrodes (one shown as 1402) formed on electrode structures 1416. Sensor assembly 1414B-0/1 can be a 3 × 4 matrix, while sensor assembly 1414B-1 can be a 3 × 3 matrix.

In this way, systems can take the form of structure that attach to a body and position electrodes over an artery location for non-invasive, convenient, continuous and/or periodic APW sensing.

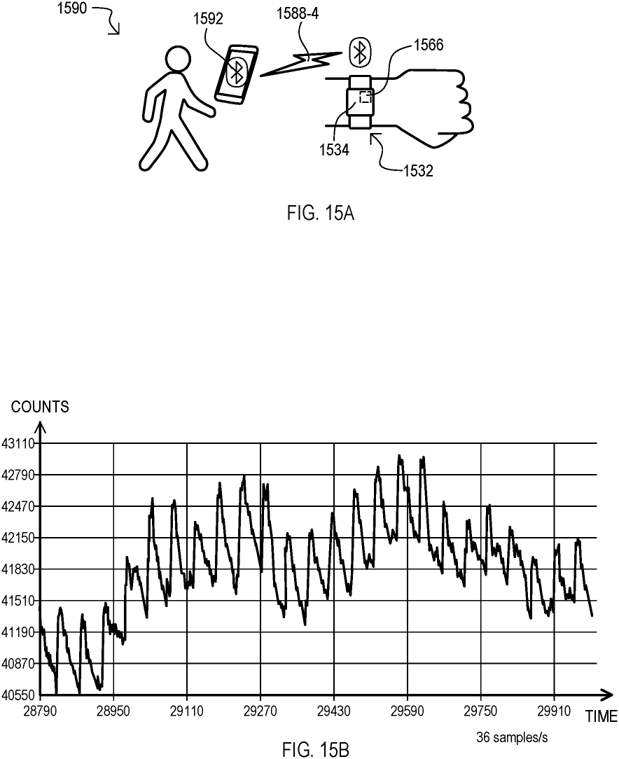

FIG. 15A is a diagram showing an APW monitoring system 1590 according to an embodiment. A monitoring system 1590 can include a APW sensing system 1532 and a host device 1592. An APW sensing system 1532 can take the form of any of those described herein, including a bracelet type system, like that shown in FIG. 14A. APW sensing system 1532 can include an APW sensing device 1534 and sensor assembly (not shown). APW sensing device 1534 can include wireless communication circuits 1566 that can transmit APW data over a wireless connection 1588-4 to host device 1592. While FIG. 15A shows a Bluetooth type wireless connection, alternate embodiments can include any other suitable communication path type.

A host device 1592 can receive APW data from APW sensing device 1534. In some embodiments, a host device 1592 can present APW related data on a display. As but one example, a pulse rate and blood pressure can be displayed. In some embodiments, a host device 1592 can include a graphical user interface (GUI) to enable a user to analyze received APW data. However, alternate embodiments can present such data in text or other forms. Further, a host device 1592 can further process APW data and/or analyze such data. Such analysis can include generating alarms in the event APW data exceeds one or more predetermined limits (e.g., pulse rate maximum and/or minimum (max/min), blood pressure maximin, irregular pulse rate, or other deviations from an expected APW). A host device 1592 can take any suitable form, including a smartphone, tablet device or other computer system, including a server system.

FIG. 15B is a diagram showing one example of GUI data that can be included on a host device. GUI data can be a graph showing counts (y-axis) for given samples over time (i.e., 36 samples per second). From such data, a pulse rate and blood pressure can be derived and presented. It is understood that FIG. 15B is but one of numerous possible GUI data presentations.

In this way, a monitoring system can include a non-invasive APW sensor attachable to a body, which can transmit APW related data to a host device, for display and/or processing.

While the devices and systems described herein have disclosed various methods according to embodiments, additional methods will now be described with reference to flow diagrams.

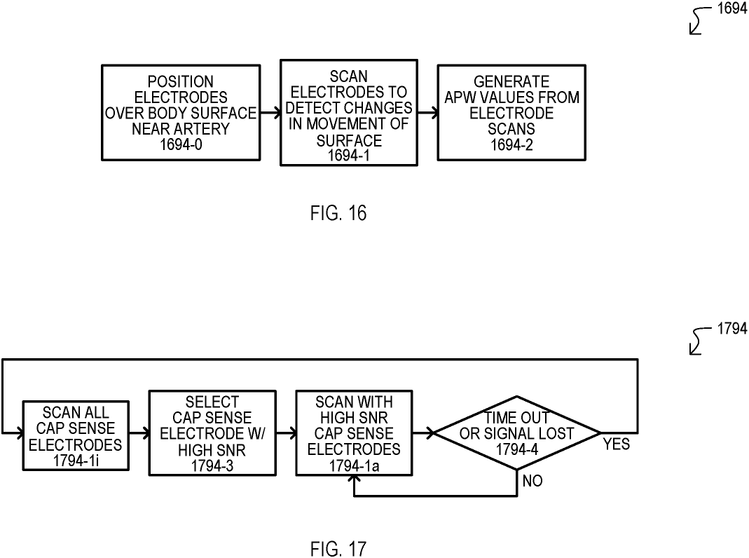

FIG. 16 is a flow diagram of a method 1694 according to an embodiment. A method 1694 can include positioning electrodes over a body surface near an artery 1694-0. Such an action can include positioning electrodes for contact or contactless sensing. Further, such an action can include positioning electrodes designed for any suitable sensing method, including capacitance, inductance or resistance sensing. In some embodiments, such an action can include attaching an APW sensing device to a location on a person’s body.

A method can include scanning electrodes to detect changes in movement of a body surface 1694-1. Such an action can include sensing according to any of the techniques disclosed herein or equivalents. Further, such an action can include scanning to acquire multiple data sets

APW values can then be generated from the electrode scans 1694-2. Such an action can include generating an entire APW waveform and/or waveform related data. Such an action can include, but is not limited to, modifying values to account for drift, and determining maximums, minimums, including local maximums, local minimums and slopes.

In this way, an APW or related data can be detected with electrodes over a body surface that sense movement caused by blood flowing through an artery.

FIG. 17 is a flow diagram of a method 1794 according to another embodiment. A method 1794 can include scanning all electrodes of a capacitance sensing (cap sense) system 1794-1i. Such an action can include sensing a self-capacitance of each electrode or a mutual capacitance between two electrodes.

Electrodes having a high signal-to-noise (SNR) ratio can be selected 1794-3. Such an action can include subjecting scanned electrode values to a SNR analysis. SNR values for each electrode can be compared to one or more limits to determine which electrodes are high SNR electrodes. Such limits can be established in any suitable manner, including being predetermined limits, or limits determined according to values of the sampled data set.

A scan can be made with high SNR cap sense electrodes 1794-1a. Such an action can include multiple scans over time. In some embodiments, such an action can include scanning over time period sufficient to acquire APW data, including scanning over multiple APWs. Scanning with high SNR electrodes 1794-1a can continue while a scan timeout period has not been exceeded or the signal has not been lost (NO from 1794-4). If a scan timeout period has been reached or the signal lost (YES from 1794-4), a method 1794 can return to scanning all electrodes 1794-1i.

In this way, a method can use capacitance sensing to detect APW and related data with only high SNR electrodes from a set of electrodes.

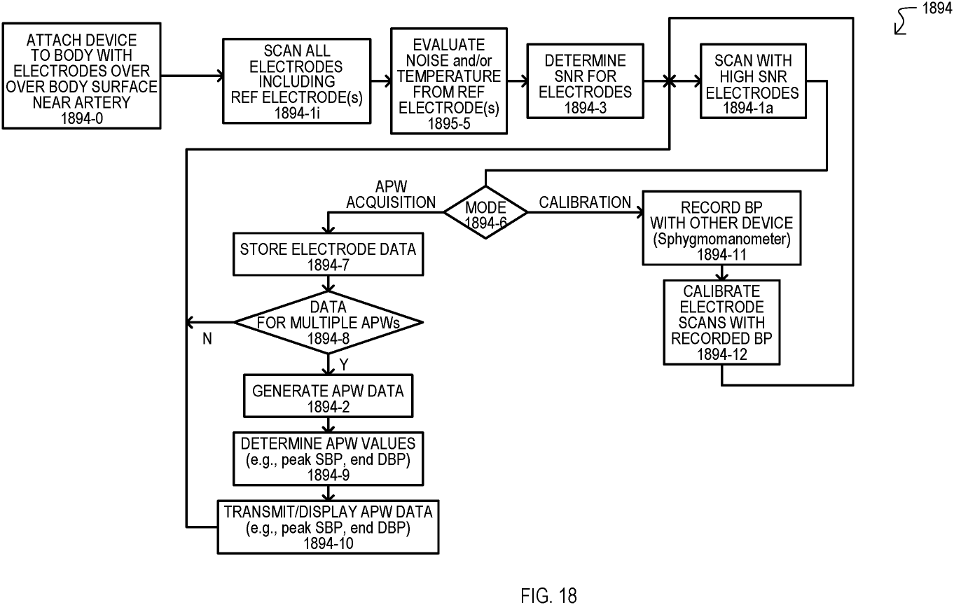

FIG. 18 is a flow diagram of a method 1894 according to another embodiment. A method 1894 can include calibrating APW sensing electrodes with another device, acquiring APW data with such electrodes, and transmitting/ displaying APW data. A method 1894 can include attaching a device to a body with electrodes over a body surface near an artery 1894-0. Such an action can include any of those described herein and equivalents. Electrodes can be scanned, including a reference electrode 1894-1i. Such an action can include acquiring data (e.g., counts) for each electrode according to any of the techniques described herein or equivalents (e.g., capacitance sensing, inductance sensing, resistance sensing). A reference electrode can be an electrode included for detecting sensing conditions as disclosed in other embodiments. A reference electrode can be a dedicated electrode (i.e., used only as a reference electrode) or dual purpose electrode (i.e., used for sensing in some configurations).

Sensing conditions can be evaluated with a reference electrode 1894-5. In some embodiments, this can include determining a temperature and/or noise condition. An SNR for each electrode can be determined 1894-3. In some embodiments, such an action can use noise conditions from a reference electrode. A scan with high SNR electrodes can be performed 1894-1a. Such actions (1894-3/1a) can include any of those described herein or equivalents.

A method 1894 can have different actions depending upon a mode 1894-6. Such a mode can be established by a user or can be automatic depending upon a system state (e.g., a method executes calibration upon power-up and/or reset). In a sensing mode (APW acquisition from 1894-6), electrode data can be stored 1894-7. Such an action can include storing data in a volatile or nonvolatile fashion.

A method 1894 can continue to scan with high SNR electrodes (N from 1894-8) until data has been acquired for multiple APWs (Y from 1894-8). With data for multiple APWs, a method 1894 can generate APW data 1894-2. Such an action can include any of those described herein or equivalents. In the embodiment shown, particular APW values (e.g., any of those shown in FIG. 1B) can be determined from the APW data 1894-9. Such particular APW values, as well as an APW waveform can be transmitted to another device and/or displayed on another device 1894-10. Any of actions 1894-2/9/10 can be performed by a device that executes the electrode scanning, or can be executed by another device that receives raw scan data.

In a calibration mode (CALIBRATION from 1894-6), a method 1894 can include recording APW data with another device 1894-11. Such an action can include using another device (e.g., sphygmomanometer) to record APW data. In some embodiments, such calibration data can be recorded while high SNR electrodes are scanned. In other embodiments, such calibration data can be recorded before high SNR electrodes are scanned. Electrode scan data can be calibrated using recorded calibration data 1894-12. After such calibration 1894-12, a method 1894 can return to scanning (1894-1a).

In this way, a method can scan for APW data with high SNR data, and calibrate such scans with data from another APW sensing device.

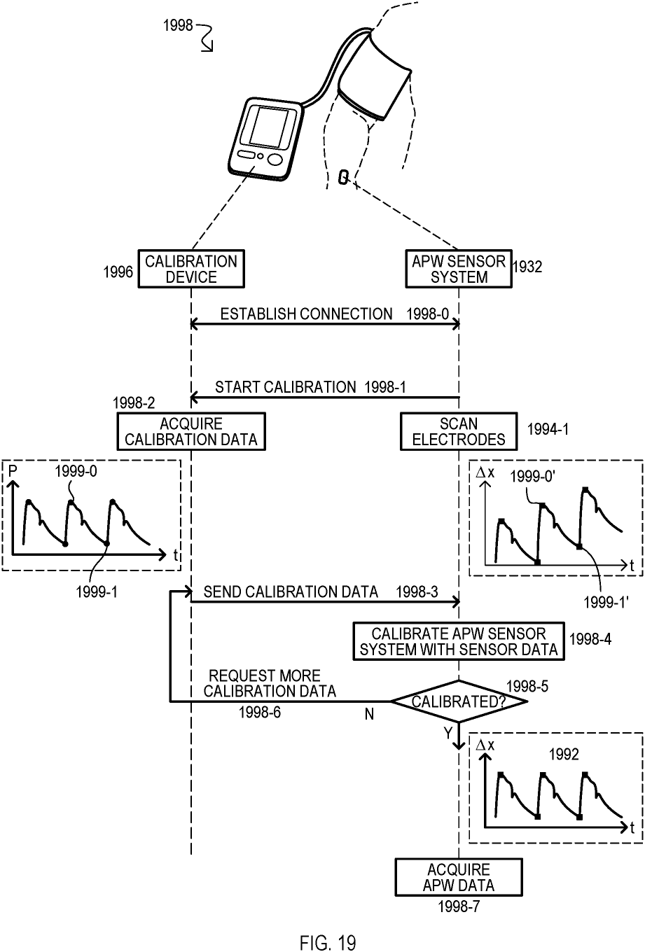

FIG. 19 is a diagram showing a calibration method 1998 according to an embodiment. APW sensing systems may provide readings that can vary for each different application. Such variance can result from factors including but not limited to: environment, sensor orientation, sensor position, location on body, or subject physiology. Accordingly, an APW sensor system can benefit from an initial calibration with a calibrating device. FIG. 19 shows a calibration system and method 1998 according to one embodiment.

A calibration system 1998 can include an APW sensor system 1932 and a calibration device 1996. An APW sensor system 1932 can take the form of any of those described herein, or an equivalent. A calibration device 1996 can sense the same, or related APW features, as the APW sensor system 1932. However, a calibration device 1996 can provide initial results that can be more accurate than an uncalibrated APW sensor system 1932. A calibration device 1996 and APW sensor system 1932 can be in communication with one another over any suitable connection, including a wired or wireless connection. In one embodiment, a calibration device 1996 can be a sphygmomanometer, and a APW sensor system 1932 can utilize capacitance sensing.

Referring still to FIG. 19, a method 1998 can include establishing a connection 1998-0 between the calibration device 1996 and the APW sensor system 1932. Once communication between the two devices (1996/1932) has been established, a calibration operation can start 1998-1. Such an action can include a calibration device 1996 acquiring calibration data 1998-2, and the APW sensor system 1932 scanning electrodes of its sensor assembly 1994-1. In some embodiments, such actions (1998-2/1994-1) can include calibration device 1996 and APW sensor system 1932 acquiring data over a same time period. Calibration data can provide values for adjusting how APW sensor system 1932 acquires and/or processes sensor data from scanned electrodes. In some embodiments, calibration data can indicate particular points in a waveform corresponding to a feature. In one embodiment, calibration data can be for a blood pressure waveform, and can indicate a systolic peak 1999-0 and well as a diastolic pressure end 1999-1. Sensor data acquired in 1994-1 can result in an initial waveform that varies from a desired waveform. In one embodiment, sensor data can be for an APW, and can sense a systolic peak 1999-0′ and well as a diastolic pressure end 1999-1′. However, such initial data points may be offset from a desired waveform.

A method 1998 can include an APW sensor system 1932 sending calibration data to a sensor device 1998-3. From calibration data, an APW sensor system 1932 can perform a calibration operation 1998-4 that can adjust how electrode scan data is generated and/or processed. In one embodiment, calibration data can indicate corresponding points in sensor data, enabling an APW sensor system 1932 to derive a function and/or offset to arrive to arrive at desired sensor results 1999-2.

If calibration is not successful (N from 1998-5), an APW sensor system 1932 can request more calibration data 1998-6. If calibration is successful (Y from 1998-5), an APW sensor system 1932 can acquire APW data 1998-7. Such an action can include any of those described herein or equivalents.

Embodiments can include systems, methods and devices having one or more electrodes and a sensor structure configured to position electrodes over a surface of a body that includes an artery. A capacitance sensing circuit can be coupled to the electrodes and configured to acquire capacitance values of the electrodes over a predetermined time period. The capacitance values can correspond to a distance between the body surface and the at least one electrode. Processor circuits can be configured to generate APW data from the capacitance values.

Embodiments can include systems, methods and devices that include, by operation of a sensor structure, positioning at least one electrode over a body surface proximate an artery; over a predetermined time period, sensing capacitance values for the at least one electrode; storing the capacitance values in a memory; and generating arterial pressure waveform (APW) data from the stored capacitance values.

Embodiments can include systems, methods and devices that include a plurality of input/output (IO) connections coupled to a substrate; capacitance sense circuits formed with the substrate and configured to generate capacitance values for at least one of the IOs; memory circuits formed with the substrate and configured to store the capacitance values; and processor circuits formed with the substrate and configured to generate arterial pressure waveform (APW) from the stored capacitance values.

Systems, methods and devices according to embodiments can further include the at least one electrode comprising an array of electrodes; and capacitance sensing circuits acquiring capacitance values for each electrode of the array.

Systems, methods and devices according to embodiments can further include a sensor structure that includes a compressible high permittivity material configured to be positioned between the at least one electrode and the surface of the body.

Systems, methods and devices according to embodiments can further include a plurality of electrodes, and a noise sensing circuit configured to determine a SNR for each electrode. Processor circuits can be configured to exclude capacitance values for electrodes having SNRs below a predetermined limit from the generation of APW data.

Systems, methods and devices according to embodiments can further include a plurality of electrodes, and a noise sensing circuit configured to determine a SNR for each electrode. Electrode selection circuits can be configured to, in a first sensing operation, connect each electrode of the plurality of electrodes the capacitance sensing circuit, and in a second sensing operation, exclude electrodes having a SNR below a predetermined threshold from being connected to the capacitance sensing circuit.

Systems, methods and devices according to embodiments can further include a capacitance sensing circuit configured to sense a self-capacitance of the at least one electrode.

Systems, methods and devices according to embodiments can further include a plurality of electrodes. A capacitance sensing circuit can be configured to sense a mutual capacitance between at least two of the electrodes.

Systems, methods and devices according to embodiments can further include a sensor structure including a band having the at least one electrode disposed on an inner surface of the band.

Systems, methods and devices according to embodiments can further include a sensor structure including an adhesive configured to attach the sensor structure to a body surface.

Systems, methods and devices according to embodiments can further include at least one display configured to display a APW from the APW data.

Systems, methods and devices according to embodiments can further include positioning an array of electrodes over the body surface; and sensing capacitance values includes sensing a capacitance of each of the electrodes.

Systems, methods and devices according to embodiments can further include mapping capacitance values to blood pressure values with blood pressure readings from another device.

Systems, methods and devices according to embodiments can further include generating APW data, including determining local minima and maxima for the capacitance values.

Systems, methods and devices according to embodiments can further include capacitance sense circuits having sigma-delta ADC circuits and multiplexer circuits configured to selectively connect the IOs to the sigma-delta ADC circuit.

Systems, methods and devices according to embodiments can further include wireless circuits formed with the substrate and configured to wirelessly transmit the APW data from the device to another device.

Systems, methods and devices according to embodiments can further include the capacitance sense circuits configured to detect noise on at least one of the IOs. Processor circuits can be configured to determine a SNR for each IO with respect to the APW. Selection circuits can be formed with the substrate and configured to couple all IOs to the capacitance sense circuits in a SNR sensing operation, and couple IOs having a SNR above a predetermined threshold to generate capacitance values for generating the APW values.

It should be appreciated that reference throughout this specification to “one embodiment” or “an embodiment” means that a particular feature, structure or characteristic described in connection with the embodiment is included in at least one embodiment of the present invention. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the invention.

Similarly, it should be appreciated that in the foregoing description of exemplary embodiments of the invention, various features of the invention are sometimes grouped together in a single embodiment, figure, or description thereof for the purpose of streamlining the disclosure aiding in the understanding of one or more of the various inventive aspects. This method of disclosure, however, is not to be interpreted as reflecting an intention that the claims require more features than are expressly recited in each claim. Rather, inventive aspects lie in less than all features of a single foregoing disclosed embodiment. Thus, the claims following the detailed description are hereby expressly incorporated into this detailed description, with each claim standing on its own as a separate embodiment of this invention.

While this invention has been described with reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A system, comprising:
at least one electrode;
a sensor structure configured to position the at least one electrode over a surface of a body that includes an artery;
a capacitance sensing circuit coupled to the at least one electrode and configured to acquire capacitance values of the at least one electrode over a predetermined time period, the capacitance values corresponding to a distance between the body surface and the at least one electrode; and
processor circuits configured to generate arterial pressure waveform (APW) data from the capacitance values.

2. The system of claim 1, wherein:
the at least one electrode comprises an array of electrodes; and
the capacitance sensing circuit acquires capacitance values for each electrode of the array.

3. The system of claim 1, wherein:
the sensor structure includes a compressible high permittivity material configured to be positioned between the at least one electrode and the surface of the body.

4. The system of claim 1, further including:
the at least one electrode comprises a plurality of electrodes;
a noise sensing circuit configured to determine a signal-to-noise ratio (SNR) for each electrode; and
the processor circuits are configured to exclude capacitance values for electrodes having SNRs below a predetermined limit from the generation of APW data.

5. The system of claim 1, wherein:
the at least one electrode comprises a plurality of electrodes;
a noise sensing circuit configured to determine a signal-to-noise ratio (SNR) for each electrode; and
electrode selection circuits configured to
in a first sensing operation, sequentially connect electrodes the capacitance sensing circuit, and
in a second sensing operation, exclude electrodes having a SNR below a predetermined threshold from being connected to the capacitance sensing circuit.

6. The system of claim 1, wherein:
the capacitance sensing circuit is configured to sense a self-capacitance of the at least one electrode.

7. The system of claim 1, wherein:
the at least one electrode comprises a plurality of electrodes; and
the capacitance sensing circuit is configured to sense a mutual capacitance between at least two of the electrodes.

8. The system of claim 1, wherein:
the sensor structure comprises a band having the at least one electrode disposed on an inner surface of the band.

9. The system of claim 1, wherein:
the sensor structure comprises an adhesive configured to attach the sensor structure to the body surface.

10. The system of claim 1, further including:
at least one display configured to display an APW from the APW data.

11. A method, comprising:
by operation of a sensor structure, positioning at least one electrode over a body surface proximate an artery;
over a predetermined time period, sensing capacitance values for the at least one electrode;
storing the capacitance values in a memory; and
generating arterial pressure waveform (APW) data from the stored capacitance values.

12. The method of claim 11, wherein:
positioning at least one electrode includes positioning an array of electrodes over the body surface; and
sensing capacitance values includes sensing a capacitance of each of the electrodes.

13. The method of claim 11, further including:
mapping capacitance values to blood pressure values with blood pressure readings from another device.

14. The method of claim 11, further including:
generating APW data includes determining local minima and maxima for the capacitance values.

15. A device, comprising:
a plurality of input/output (IO) connections coupled to a substrate;
capacitance sense circuits formed with the substrate and configured to generate capacitance values for at least one of the IOs;
memory circuits formed with the substrate and configured to store the capacitance values; and
processor circuits formed with the substrate and configured to generate arterial pressure waveform (APW) data from the stored capacitance values.

16. The device of claim 15, wherein:
the capacitance sense circuits are configured to sense a self-capacitance of the at least one IO.

17. The device of claim 15, wherein:
the capacitance sense circuits are configured to sense a mutual capacitance between at least two of thelOs.

18. The device of claim 15, wherein:
the capacitance sense circuits comprises a sigma-delta analog-to-digital conversion (ADC) circuits; and
multiplexer circuits configured to selectively connect the IOs to the sigma-delta ADC circuit.

19. The device of claim 15, further including:
wireless circuits formed with the substrate and configured to wirelessly transmit the APW data from the device to another device.

20. The device of claim 15, further including:
the capacitance sense circuits are configured to detect noise on at least one of the IOs;
the processor circuits are further configured to determine a signal-to-noise ratio (SNR) for each IO with respect to the APW; and
selection circuits formed with the substrate and configured to
couple all IOs to the capacitance sense circuits in a SNR sensing operation, and
couple IOs having a SNR above a predetermined threshold to generate capacitance values for generating the APW values.
